(19) United States
(12) Patent Application Publication
(10) Pub. No.: US 2023/0402129 A1
(43) Pub. Date: Dec. 14, 2023

(54) A METHOD OF EPITOPE-BASED VACCINE DESIGN

(71) Applicants: The University of British Columbia, Vancouver (CA); The James Hutton Institute, Dundee (GB)

(72) Inventors: Wilfred Jefferies, Surrey (CA); Paolo Ribeca, Midlothian, Scotland, Edinburgh (GB)

(21) Appl. No.: 18/036,375

(22) PCT Filed: Nov. 23, 2021
(86) PCT No.: PCT/CA2021/051666

§ 371 Date: May 10, 2023

Related U.S. Application Data
(60) Provisional application No. 63/117,496, filed on Nov. 24, 2020.

(51) Int. Cl. G16B 35/20; G16B 20/40; G16B 40/00

(52) U.S. Cl. CPC G16B 35/20; G16B 20/40; G16B 40/00

(57) ABSTRACT

The present invention provides a method of epitope-based vaccine design. The method comprising: a) providing one or more target amino acid sequence(s); b) optionally discarding peptides based on one or more predetermined features; c) parsing and selecting MHC I and/or MHC II binding peptides from said sequences; and d) assembling the MHC I and/or MHC II binding peptides optionally with linkers to produce an assembly of peptides.

### FIELD OF THE INVENTION

This invention pertains generally to methods of vaccine design and, more particularly a method of epitope-based vaccine design.

### BACKGROUND OF THE INVENTION

An outbreak of pneumonia like disease termed COVID-19 caused by a novel coronavirus, SARS-CoV-2, has spread across the world and become a global pandemic with more than 258 million confirmed cases and 5.1 million deaths worldwide. While potential therapies for the treatment of COVID-19 are being investigated, in the absence of an effective SARS-CoV-2 vaccine, the probability of containment is low and the global pandemic will continue. First generation vaccines targeting SARS-CoV-2 have been developed by Pfizer, Moderna and others and, to date, over 52% of the world's population has been administered at least one dose of vaccine. These first generation vaccines all target spike protein with the Oxford vaccine using an adenoviral vector and the vaccines by Moderna and Pfizer are RNA based.

A number of SARS-CoV-2 variants have been identified, including those with mutant Spike proteins. While the currently approved vaccines are effective at limiting the severity of the disease and currently there are no variants able to completely circumvent this property, some variants can partially evade existing vaccines and no vaccines confers long-lasting, sterilizing immunity. In general, spike is hypervariable and prone to mutations; accordingly, it is possible that a variant able to generate more serious cases or completely evade the vaccine will arise in the future. Moreover, a low number of antibodies from survivors (10-30%) target spike protein. In addition, vaccines targeting spike protein will not immunise all individuals due to individual HLA variability.

To work, a human vaccine must contain pathogen-derived molecules (typically proteins or glycans) processed first by the host cell into peptides and then presented (antigen presentation) at the host cell surface on its Human Leukocyte Antigens (HLA). Similar mechanisms operate in most other organisms, albeit the genes involved in the immune response might be different. Human HLAs are encoded within the highly polymorphic major histocompatibility complex (MHC) on Chromosome 6. This process leads to presentation of peptides originating from self and pathogen. HLA-peptide complexes are then specifically recognized by T-cells via the T-cell receptor (TCR); while self-peptide ligands do not typically elicit a response from the immune system, an immunogenic foreign peptide ligand-HLA complex will bind a TCR and trigger an immune response that leads to the development of cytotoxic and memory T- and B-cells. There are many components in the host immune system, but HLA molecules are the chief determinants of antigen presentation to the T-cells for subsequent activation of the immune response.

The classical HLA genes are the most polymorphic genes in the human genome, with some having more than a thousand known alleles, that are distributed unevenly around the world but typically clustered according to ethnicity. These genes are divided into two subgroups, mainly based on the source of the peptides they tend to present: HLA I genes are expressed on all cells except red blood cells, and present peptides of intracellular origin (e.g. from self or viruses), whereas HLA-II genes are expressed only on professional antigen presenting cells and present peptides that originate extracellularly (e.g. from bacteria). Thus, the response to a viral pathogen such as SARS-CoV-2 is mediated by HLA-1.

The HLA genes are co-dominantly expressed and encode HLA proteins that are referred to as HLA Class I (HLA-A, -B, -C,) and HLA Class II (HLA-D). They are critical in priming adaptive immune responses. CD4+ T helper/inducer cells recognize viral peptides bound to HLA-II encoded proteins and CD8+ Cytotoxic T cells recognize viral peptides bound to HLA-A, -B, -C, encoded proteins. The vast polymorphism in their extracellular peptide binding domains leads to the diversity of peptide antigens that can be bound and subsequently recognized by T cell receptors. HLA class I molecules generally present short (8-12 amino acids) intracellularly-derived peptides, such as viral antigens. HLA class II molecules are capable of presenting longer (i.e., generally more than 13 amino acids) extracellularly derived peptides, such as antigenic fragments generated from viral proteins. The HLA allele polymorphism renders each variant protein a distinct product with the main difference focused on the peptide-binding groove and the conformation of adjacent regions directly engaged with peptide binding and interaction with the TCR. A T-cell will recognize bound antigen as a complex with a restricted allelic variant of HLA molecule. Depending on the combinations of HLA-1 and II alleles expressed, an individual may be differently equipped to resist certain viruses, including coronaviruses. Thus, individual genetic variation across HLA genes aids in understanding how variation in HLA may affect the course of COVID-19, and could help identify individuals at higher risk of succumbing to the disease.

Previous research on related virus strains (SARS-CoV-1 and MERS-Cov) demonstrated that viral antigen presentation of SARS-CoV-1 mainly depends on HLA-I and HLA-II molecules. Numerous HLA-I polymorphisms correlate to susceptibility of SARS-CoV-1, such as HLA-B*46:01 5, HLA-B*07:03, HLA-DR B1*12:02, and HLA-Cw*08:01, whereas the HLA-DR*03:01, HLA-Cw15:02 and HLA-A*02:01 alleles are related to the protection from SARS-CoV-1 infection 7. HLA-II molecules, such as HLA-DRB1*11:01 and HLA-DQB1*02, are associated with the susceptibility to MERS-CoV infection. These data suggest that individual HLA genotypes may differentially control susceptibility or protection in T-cell mediated anti-SARS-CoV-2 responses. Thus, HLA polymorphism could potentially alter disease outcomes and SARS-CoV-2 transmission. The enormous diversity in HLA genes means that some individuals can present an antigen and mount a strong immune response against it, while others cannot present it at all. This is especially relevant for vaccination strategies involving subunit vaccines, since the number of available antigens can be very small. In fact, HLA polymorphism is a likely basis for the observed variations in vaccine efficacy.

Epitope-based or string of beads vaccines use concatemers of short immunogenic peptide sequences derived from antigens that are recognised by either CD4 or CD8 T-cells in the context of HLA-II or HLA-I respectively. They have several advantages over whole attenuated or subunit vaccines because they do not contain potentially infectious material. Furthermore, peptides can be chosen to take the genetic variation of pathogens and HLA-binding specificities into account. Development of such vaccines requires bioinformatics for prediction of HLA epitopes. Machine-learning methods, such as probabilistic models, neural networks, and support vectors machines, are routinely used with high accuracy for epitope prediction. Different algorithms have been used to create string of bead vaccines that generally concentrate on binding peptides for a small number of HLA-I epitopes.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a method of epitope-based vaccine design. In accordance with an aspect of the present invention, there is provided a method of epitope-based vaccine design comprising: providing target amino acid sequence(s) and annotation; a list of genomic features to be excluded from the result; a list of alleles which describe the target population; filtering peptides based on their antigenic score; filtering peptides based on their similarity with human peptides; filtering peptides based on a database of known genomic features (such as secondary structure or other); visualising the filtering process graphically in order to do quality control; for all the selected peptide lengths: predict optimal MHC-I and MHC-II peptides based on population structure; selecting peptides based on the p-value returned by the predictor; recombining peptides produced for different peptide lengths; generating statistics on: the number and lengths of peptides needed for each gene and the alleles in the population covered by each peptide; optionally selecting the peptides coming from “denser” genes (i.e., the ones providing protection to most alleles in the population with a shorter overall peptide length) in order to compact the construct; and assembling the peptides optionally with linkers.

In accordance with an aspect of the present invention, there is provided a method of epitope-based vaccine design comprising: a) providing one or more target amino acid sequences; b) optionally discarding peptides based on one or more predetermined features; c) parsing and selecting peptides recognized by the immune system of an organism; and d) assembling such peptides optionally with linkers to produce an assembly of peptides. In certain embodiments, the organism is human and the peptides are MHC I and/or MHC II binding peptides. In certain embodiments, the organism is a species relevant to farming or another human activity.

In certain embodiments of the above method step (c) comprises: (i) providing one or more MHC I and/or MHC II alleles which meet a pre-determined frequency threshold in a population to be vaccinated, optionally the one or more MHC I and/or MHC II alleles have a frequency greater than 1% in the population; and (ii) selecting peptides that bind to one or more of said one or more MHC I and/or MHC II alleles having a threshold binding efficiency. Optionally further comprising: (iii) merging any overlapping peptides selected in step (ii) to produce said MHC I and/or MHC II binding peptides.

In certain embodiments, the one or more predetermined features comprise region having low antigenic score, region of hypervariability in a population of said one or more pathogens and/or regions having significant similarity to proteins in the population, optionally wherein the population is human.

In certain embodiments, the one or more target amino acid sequences are from any protein or peptide which meets the selection criteria of the method.

In certain embodiments, the one or more amino acid sequences encoded by genes of interest from one or more pathogens are consensus sequences or sequences from one or more strains of said one or more pathogens. In certain embodiments, peptides arising from hypervariable regions (i.e., genomic regions where variants can be found for a number of strains of said one or more pathogens) are discarded, so as to guide the immune systems to recognize peptides that are conserved across most pathogen strains.

In certain embodiments, the one or more pathogens are selected from one or more strains of SARS-CoV-2 and/or one or more strains of influenza. In certain embodiments, the targeted peptides arise from one or more aberrant, mutated, defective, toxic or neoplastic proteins.

In certain embodiments, the method further comprises constructing a construct which expresses the assembly of peptides, optionally the construct is a SAM RNA construct.

### BRIEF DESCRIPTION OF THE DRAWINGS

These and other features of the invention will become more apparent in the following detailed description in which reference is made to the appended drawings.

FIG. 1 defines the inputs of a workflow of the epitope-based vaccine design method of the embodiment illustrated in FIG. 5.

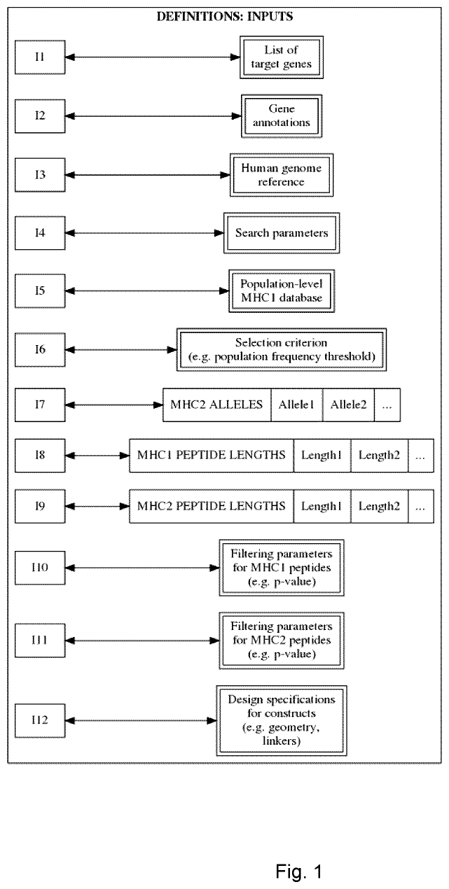

FIG. 2 defines the tasks performed during a workflow of the epitope-based vaccine design method of the embodiment illustrated in FIG. 5.

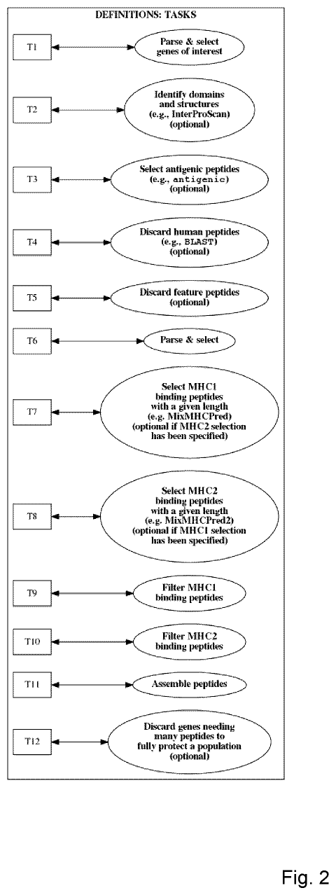

FIG. 3 illustrates the intermediate results of a workflow of the epitope-based vaccine design method of the embodiment illustrated in FIG. 5.

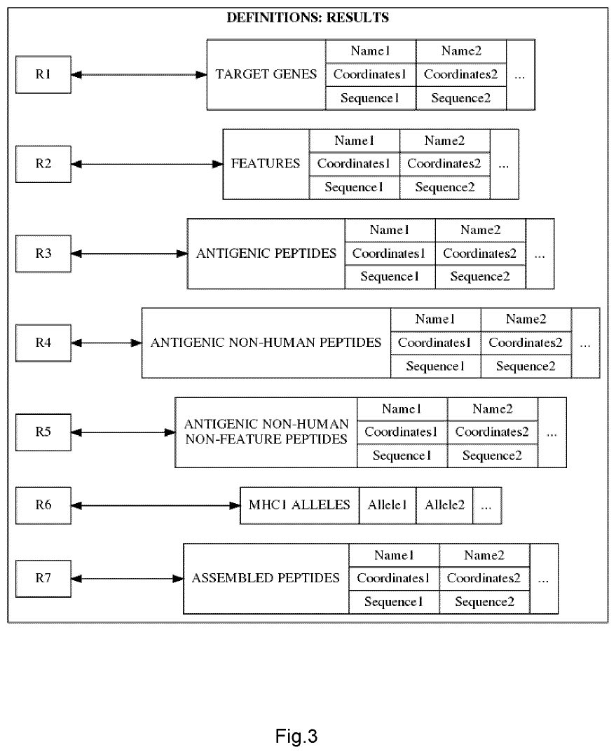

FIG. 4 defines the outputs of a workflow of the epitope-based vaccine design method of the embodiment illustrated in FIG. 5.

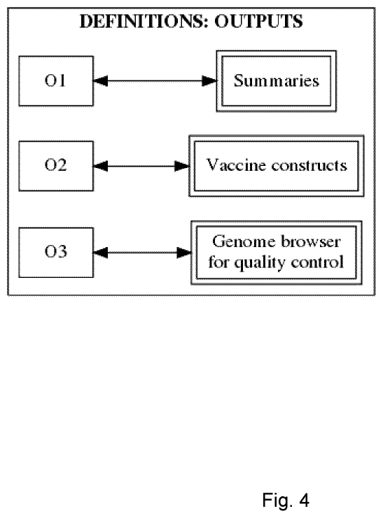

FIG. 5 defines the overall workflow of the epitope-based vaccine design method of an embodiment the invention. The blocks set forth in FIG. 5 are as defined in FIGS. 1 to 4.

FIG. 6 illustrates the predicted effectiveness at protecting each human MHC class I/II immunotype against each Covid19 gene for two different constructs having a different length—whenever a matrix cell is red there is in the construct at least one immunogenic peptide predicted for the corresponding immunotype and viral gene. Viral genes are presented in random order, and the order is different for the two figures. One can see how even with a small construct it is possible to find immunogenic peptides for most viral genes and most immunotypes. FIG. 6 also illustrates the value of exploring with Jennerator the space of possible vaccine constructs in order to maximize the number of protected viral genes and immunotypes with respect to the length of the construct.

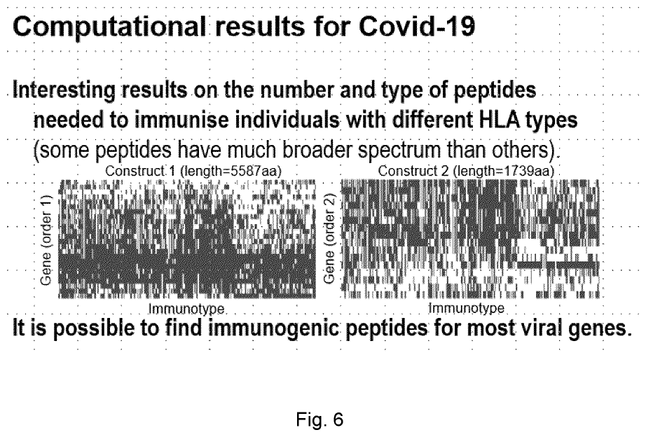

### DETAILED DESCRIPTION OF THE INVENTION

The present invention provides an in silico method of epitope-based vaccine design. In particular, the present invention provides a method of predicting epitopes for use in epitope-based vaccines. The methods of the present invention may be used in the development of vaccines for human and non-human populations. In certain embodiments, the methods of the present invention are used in the development of vaccines for human populations. In certain embodiments, the methods of the present invention are used in the development of non-human populations, including non-human mammals such as canines, felines, equines, bovines and non-human primates.

The method may be used to design vaccines for a variety of targets including but not limited to pathogens and neoplasms. Exemplary pathogens include but are not limited to bacterial pathogens or viral pathogens, including respiratory viral pathogens such as influenza and coronavirus such as SARS-CoV-2. The methods of the present invention may be used in the development of a vaccine for a single target (i.e. a single type of pathogen such as a coronavirus including but not limited to SARS-CoV-2 or influenza) or multiple targets (i.e. multiple types of pathogens such as multiple strains of coronavirus or influenza or a vaccine that targets both coronavirus and influenza).

In certain embodiments, the epitopes are selected from active sites of proteins required for pathogen replication or function.

In certain embodiments, the epitopes are from proteins that have been mutated in cancer.

In certain embodiments, the epitopes are from prion proteins or amyloid precursor proteins.

In certain embodiments, the epitopes are from defective ribosomal products (DRiPs).

In certain embodiments, the epitopes are from toxic proteins (for instance, toxins produced by viruses, bacteria, animals or plants).

In certain embodiments, the methods of epitope-based vaccine design discard potential epitopes from the target(s) having significant similarity to proteins in the population to be vaccinated. By not including such epitopes in the vaccine, the vaccine may appear to be “more foreign” to the immune system of the population to be vaccinated and thereby potentially enhancing vaccine efficacy.

Accordingly, in certain embodiments there is provided a method to predict T-cell MHC-I (e.g. HLA-I for human specific vaccines) and/or MHC-II (e.g. HLA-II for human specific vaccines) epitopes of specific targets for development of target-specific vaccines. In certain embodiments, methods to predict epitopes relevant to the immune system of non-human species (for instance, animals relevant to farming or other human activities) are provided.

In certain embodiments there is provided a method to predict T-cell MHC-I (e.g. HLA-I for human specific vaccines) and/or MHC-II (e.g. HLA-II for human specific vaccines) epitopes of specific pathogens for development of pathogen-specific vaccines. In specific embodiments, there is provided a method to predict T-cell MHC-I (e.g. HLA-I) and/or MHC-II (e.g. HLA-II) epitopes of SARS-CoV-2 for development of SARS-CoV-2-vaccines. In certain embodiments there is provided a method to predict T-cell MHC-I (e.g. HLA-I) and/or MHC-II (e.g. HLA-II) epitopes of specific neoplasms for development of neoplasm-specific vaccines. Also provided are methods of designing and producing epitope-based vaccines.

In certain embodiments, the methods of the present invention may be used to design vaccines tailored to produce a TH1 or TH2 response.

The methods of the present invention may be used to generate epitopes for one or more targets. The targets may be one or more neoplasm-specific proteins or one or more pathogens. Accordingly, in certain embodiments, the methods of the present invention may be used to generate epitopes for one or more pathogens. In certain embodiments, the methods are used to generate epitopes for more than one pathogen type. The one or more pathogens may be various strains or mutants of a particular pathogen. For example, the one or more pathogens may be various strains of coronavirus, including but not limited to SARS-CoV-2, various types of influenza viruses or a combination of different viral pathogens, such as coronavirus strains, including but not limited to SARS-CoV-2 strains and strains of influenza.

In certain embodiments, the generated epitopes are present in more than one strain of the pathogen. In certain embodiments, the generated epitopes are common epitopes (i.e. present in all identified strains of the particular pathogen). In alternative embodiments, the generated epitopes are present in a specific strain or variant of the pathogen. The designed vaccines may comprise one or more epitopes present in more than one strain of pathogen (e.g. common epitopes), one or more epitopes present in specific strain(s) of pathogen or a combination thereof.

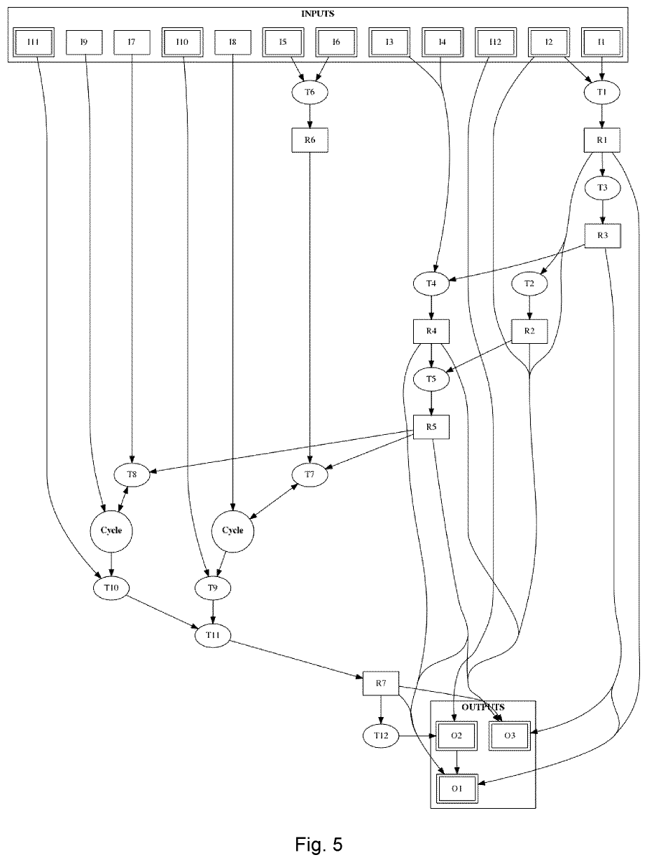

In certain embodiments, the methods of the present invention may be used to generate epitopes for a population of pathogens (for instance, a virus such as SARS-CoV-2 and all its circulating variants), as follows:
1. For some partition of the sequences (for instance, a list of sequence sets, each set corresponding to SARS-CoV-2 sequences coming from a specific country), regions in each set that are variable with respect to a common reference genome are selected and provided to the workflow of FIG. 5 as part of inputs I1. This adds to the results of the workflow the peptides originated by such variable regions, if they survive the selection process operated by the workflow, thus providing protection specific to each set of sequences (for instance, protection against SARS-CoV-2 variants specific to a list of countries of interest).
2. For a set of sequences in a population of choice (for instance, a set of SARS-CoV-2 variants), hypervariable regions of a common reference genome are selected and provided to the workflow of FIG. 5 as part of inputs I10 and I11. This eliminates from the results of the workflow peptides originated by hypervariable regions, which are unlikely to provide cross-population protection.

In certain embodiments, the methods of the present invention may be used to predict epitopes for all alleles of MHC-I and/or MHC-II with a given frequency (for instance, >1%) in a population. In such embodiments, the methods of the present invention may be used to design of vaccines which are substantially universal (i.e. immunogenic independent of the recipient's MHC alleles). In certain, embodiments the population is a human population, such as the general population of a particular geographic area (such as worldwide or a particular country). Accordingly, in certain embodiments, the methods of the present invention allow vaccines to be tailored it towards specific geographically localized MHC types thereby potentially enhancing vaccine efficacy in particular geographic areas.

In certain human populations, a list of frequent MHC-I alleles may be: A0101, A0201, A0202, A0203, A0205, A0206, A0207, A0301, A1101, A2301, A2402, A2501, A2601, A2 602, A2603, A2902, A3001, A3002, A3101, A3201, A3301, A6601, A6801, A6802, A8001, B0702, B080 1, B1301, B1302, B1401, B1402, B1501, B1502, B1510, B1517, B1518, B1801, B2702, B2704, B2703, B2705, B3501, B3502, B3503, B3508, B3701, B3801, B3901, B3906, B4001, B4002, B4101, B4402, B4 403, B4427, B4501, B4601, B4801, B4901, B5001, B5101, B5201, B5301, B5401, B5601, B5701, B580 1, C0102, C0202, C0303, C0304, C0401, C0501, C0602, C0701, C0702, C0802, C1202, C1203, C1402, C1502, C1505, C1601, G0101.

In certain human populations, a list of frequent MHC-II alleles may be: DRB1*01:01, DRB1*01:02, DRB1*01:03, DRB1*03:01, DRB1*04:01, DRB1*04:04, DRB1*04:05, DRB1*04:08, DRB1*07:01, DRB1*08:01, DRB1*10:01, DRB1*11:01, DRB1*11:04, DRB1*12:01, DRB1*13:01, DRB1*13:03, DRB1*15:01, DRB1*16:01, DRB3*01:01, DRB3*02:02, DRB4*01:01, DRB4*01:03, DRB5*01:01, DRB5*02:02, DPA1*01:03, DPA1*02:01, DPB1*03:01, DPA1*01:03, DPA1*02:01, DPB1*11:01, DPA1*01:03, DPA1*02:01, DPB1*17:01, DPA1*01:03, DPA1*02:02, DPB1*05:01, DPA1*01:03, DPB1*02:01, DPA1*01:03, DPB1*03:01, DPA1*01:03, DPB1*04:01, DPA1*01:03, DPB1*06:01, DPA1*01:03, DPB1*105:01, DPB1*126:01, DPA1*01:03, DPB1*20:01, DQA1*02:01, DQB1*02:02, DQA1*03:01, DQB1*03:01, DQA1*03:03, DQB1*03:01, DQA1*05:05, DQB1*03:01.

The methods of the present invention may be used to select a single epitope or a group of epitopes for use in vaccines. A worker skilled in the art would readily appreciate that multi-epitope vaccines may comprise concatemers of epitopes with or without intervening linker sequences. In certain embodiments, the present invention provides a method of designing a string of beads vaccines.

In certain embodiments, the method comprises the user specifying a list of desired MHC I and/or II alleles as input. In principle, each individual person only needs to match one MHC I and one MHC II epitope with one of that individual's HLA Alleles. In specific embodiments, all alleles with a frequency >1% in the general human population are chosen, so as to achieve a universal vaccine. The method then comprises several steps: (1) translating the DNA sequences for viral genes, possibly derived from the consensus from multiple viruses, into amino acid sequences (2) masking part of the resulting products, depending on the local biochemical properties of the peptides—for instance, regions with low antigenic score, or regions which are hypervariable in a population of pathogens, may be removed. Regions with significant identity to human proteins may also be removed (3) identifying a list of relevant peptides, based on the list of alleles specified in input and on several in-silico predictors (such as those in the MixMHCpred: https://bmcbioinformatics.biomedcentral.com/articles/10.1186/s12859-018-2561-z; MixMHC2pred: https://www.nature.com/articles/s41587-019-0289-6; and NetMHC1-2 families of methods: https://www.jimmunol.org/content/199/9/3360) (4) selecting the peptides thus obtained and ranking by their score or statistical significance value, and keeping the best peptides for each allele are kept (5) several rounds of prediction may be carried out, each one with different parameters for the predictor; combining the results (6) merging the best peptides for all the specified MHC alleles whenever they are overlapping, resulting in a final list of extended peptides (7) merging the peptides into a construct, either as they are or by means of interleaved linkers. Optionally, optimizing the sequence of the linkers in-silico in order to maximise the probability for the peptides to be cleaved at the position of the linkers. In specific embodiments, contigs are designed with the short protease sensitive linkers and FLAG-tag which are less than 20 kb in length.

In certain embodiments, the inputs of the method are: one or more prokaryotic or eukaryotic genomes and annotations (for instance, mammalian, fungal, bacterial, viral)—sequence and annotation can be provided at the level of single sequence, consensus sequence, or population; a list of genomic features to be excluded from the result; a list of alleles which describe the target population. In these embodiments, the workflow comprises, as illustrated in FIG. 5: filter peptides based on their antigenic score; filter peptides based on their similarity with human peptides; filter peptides based on a database of known genomic features (such as secondary structure or other); visualise the filtering process graphically in order to do quality control; for all the selected peptide lengths: predict optimal MHC-I and MHC-II peptides based on population structure; select peptides based on the p-value returned by the predictor; recombine peptides produced for different peptide lengths; generate statistics on: the number and lengths of peptides needed for each gene and the alleles in the population covered by each peptide; optionally select the peptides coming from “denser” genes (i.e., the ones providing protection to most alleles in the population with a shorter overall peptide length) in order to compact the construct; and connect peptides with linkers/assemble them in order to have them properly cleaved.

In certain embodiments, the method comprises mapping, optionally all, the predicted HLA epitopes for the entire SARS-CoV-2 genome or a portion thereof; creating a heat map of the predicted HLA epitopes in rank order of HLA allele frequency; using these maps and frequencies a string of beads of SARS-CoV-2 HLA binding epitopes likely to give protection is created. Based on statistics, nearly 100% of coverage for human populations, each individual person only needs to match one MHC I and one MHC II epitope with one of that individual's HLA Alleles. Optionally, the same steps are repeated for the major classifications of COVID-19: type I and type II and/or for the sub variants to see if they change their potential HLA binding epitopes.

In certain embodiments, the method further comprises predicting which amino acids are essential for pathogen replication and the invariant HLA binding peptides within are mapped. In specific embodiments related to development of coronavirus vaccines, the method further comprises mapping the invariant HLA binding peptides within the sequences essential to coronavirus replication. In certain embodiments, antigenicity of a protein/peptide for B cell responses and bias the T cell epitopes towards including these regions is also predicted. Any peptides that are homologous to the human genome are removed to accentuate the foreign nature of the epitopes and avoid self-immune effects.

In certain embodiments, the method further comprises assembly of the peptide epitopes with or without linker peptides between each of the epitope or between groups of epitopes. In certain embodiments, the linker peptides increase the yield of the desired antigenic peptides yielded by proteolytic digestion during antigen processing. In specific embodiments, the linker peptides comprise a protease cleavage site. Exemplary protease cleavage sites include a furin cleavage site, and trypsin cleavage site and chymotrypsin cleavage site. Linker peptides comprising protease cleavage sites may be inserted between each epitope or between groups of epitopes. Accordingly, in certain embodiments, the designed vaccine comprises a protease cleavage site between each epitope.

In certain embodiments, the method further comprises adding one or more sequences to enhance the immune response to the epitope string. In certain embodiments, one or more targeting motifs are added to the epitope string. Exemplary targeting motifs include but are not limited to endosome/lysosome (i.e. endolysosomal) motif. In certain embodiments where the vaccine comprises or encodes more than more immunogen, each immunogen may be fused to one or more targeting molecules. The targeting molecules may be the same for all immunogens in the vaccine or different.

In certain embodiments, the method further comprises adding a protein tag to the epitope string. Appropriate tags are known in the art and include but are not limited to HA-, FLAG®- or myc- or alpha tags. In specific embodiments, a FLAG-tag is inserted at the carboxy-terminal end of the constructs in order to examine protein expression in perform Western blots.

The method of the present invention may be utilized in the design of vaccines from various platforms including peptide based, viral vector-based or nucleic acid-based. In certain embodiments, the methods of the present invention the size capacity of the platform is taken into account when assembling the peptides into an epitope string.

In certain embodiments, the vaccine is viral vector-based or nucleic acid-based vaccine. In specific embodiments, the method is for use in the design of a self-amplifying (SAM) RNA platform. In certain embodiments, the method is for use in the design of DNA vectors. In such embodiments, the method for comprises constructing (optionally in silico) a construct which expresses the one or more identified epitopes.

In certain embodiments the method comprises optimizing for expression of the assembled epitopes.

In certain embodiments, the method comprises codon optimization.

In certain embodiments, the vaccine is a protein-based vaccine comprising one or more of the epitopes identified by the methods of the invention alone or in combination with one or more immunogenic proteins.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A method of epitope-based vaccine design comprising:
providing target amino acid sequence(s) and annotation; a list of genomic features to be excluded from the result; a list of alleles which describe the population to be vaccinated;
filtering peptides based on their antigenic score;
filtering peptides based on their similarity with peptides from the population to be vaccinated, wherein optionally the peptides from the population to be vaccinated are wild-type peptides;
filtering peptides based on a database of known genomic features;
visualising the filtering process graphically in order to do quality control; for all the selected peptide lengths: predict immunogenic peptides for the immune system of the species targeted by the vaccine (for human vaccines, predict immunogenic MHC-I and MHC-II peptides) based on population structure;
selecting peptides based on the p-value returned by the predictor;
recombining peptides produced for different peptide lengths;
generating statistics on: the number and lengths of peptides needed for each gene and the alleles in the population covered by each peptide; optionally selecting the peptides coming from “denser” genes in order to compact the construct; and
assembling the peptides optionally with linkers.

2. The method of claim 1, further comprises constructing a construct which expresses the assembly of peptides.

3. The method of claim 2, wherein said construct is a SAM RNA construct.

4. The method of claim 1, wherein the target amino acid sequence(s) is pathogen sequence(s)/consensus sequence(s)/population-level sequence(s) or one or more sequences mutated in neoplasia or an aberrant or defective or toxic protein such as prion or amyloid.

5. The method of claim 4, wherein is the target amino acid sequence(s) are from one or more viruses, wherein optionally said one or more viruses are one or more strains of coronavirus, one or more strains of influenza or a combination thereof.

6. The method of claim 4, wherein said pathogen comprises multiple strains of SARS-CoV-2.

7. A method of epitope-based vaccine design comprising:
a) providing one or more target amino acid sequence(s);
b) optionally discarding peptides based on one or more predetermined features;
c) parsing and selecting immunogenic peptides from said sequences (in the case of human vaccines, MHC I and/or MHC II binding peptides); and
d) assembling the immunogenic peptides optionally with linkers to produce an assembly of peptides.

8. The method of claim 7, wherein step (c) comprises:
(i) providing one or more MHC I and/or MHC II alleles which meet a pre-determined frequency threshold in a human population to be vaccinated, for instance all the MHC I and/or MHC II alleles that have a frequency greater than 1% in the population; and
(ii) selecting peptides that bind to one or more of said one or more MHC I and/or MHC II alleles having a threshold binding efficiency.

9. The method of claim 8, further comprising: (iii) merging any overlapping peptides selected in step (ii) to produce said MHC I and/or MHC II binding peptides.

10. The method of claim 7, wherein said one or more predetermined features comprise region having low antigenic score, region of hypervariability in a population of said one or more pathogens and/or regions having significant similarity to proteins in the population, optionally wherein the population is human.

11. The method of claim 7, wherein the one or more target amino acid sequences are from one or more pathogens are consensus sequences or sequences from one or more strains of said one or more pathogens or one or more proteins mutated in neoplasia.

12. The method of claim 11, wherein said one or more pathogens are selected from one or more strains of coronavirus, optionally SARS-CoV-2 and/or one or more strains of influenza.

13. The method of claim 7, further comprises constructing a construct which expresses the assembly of peptides.

14. The method of claim 13, wherein said construct is a SAM RNA construct.

15. The method of claim 1, wherein the one or more target amino acid sequences are from any protein or peptide which meets the selection criteria of the method.
